(19) United States
(12) Patent Application Publication
(10) Pub. No.: US 2011/0117557 A1
(43) Pub. Date: May 19, 2011

(54) Methods and Compositions For Diagnosis of Age-Related Macular Degeneration

(76) Inventors: Jeffrey A. Canter, Nashville, TN (US); Margaret A. Pericak-Vance, Miami, FL (US); Kylee M. Spencer, Hermitage, TN (US); Jonathan L. Haines, Brentwood, TN (US)

(21) Appl. No.: 12/934,027

(22) PCT Filed: Feb. 23, 2009
(86) PCT No.: PCT/US09/34882

§ 371 Date: Jan. 26, 2011

Related U.S. Application Data
(62) Division of application No. 61/030,460, filed on Feb. 21, 2008.

(51) Int. Cl. C12Q 1/68; G01N 33/68

(52) U.S. Cl. 435/6; 436/86

(57) ABSTRACT

The invention generally concerns methods and compositions for screening individuals for susceptibility to age-related macular degeneration (AMD). In particular, association with the various markers including complement factor H, LOC387717/ARMS2, C2/CFB, C3 and VEGF, indicates that a subject is at risk of AMD.

This application claims benefit of priority to U.S. Provisional Application Ser. No. 61/030,460, filed Feb. 21, 2008, the entire contents of which are hereby incorporated by reference.

This invention was made with government support under grant number EY012118 awarded by the National Institutes of Health. The government has certain rights in the invention.

### BACKGROUND OF THE INVENTION

1. Field of the Invention

The present invention relates generally to the fields of genetics and medicine. Specifically, the invention relates to compositions and methods for diagnosing or predicting the occurrence of age-related macular degeneration based on a subject's genotypes at several loci and their environmental exposures.

2. Description of Related Art

Age-related macular degeneration (AMD) is the leading cause of irreversible severe vision loss in Caucasians over the age of 50 (Centers for Disease Control and Prevention (CDC). While AMD is clearly a complex disorder, evidence from familial aggregation studies, twin studies, and segregation analysis all point to a significant role for genetic factors in the etiology of AMD (Klayer et al., 1998; Hammond et al., 2002; Heiba et al., 1994). Recent genetic research has focused primarily on the nuclear genome and resulted in new associations, such as those in the Complement Factor H gene (CFH) on Chromosome 1 and the LOC387715 gene on Chromosome 10, with the AMD phenotype (Haines et al. 2005; Hageman et al., 2005; Klein et al. 2005; Edwards et al., 2005; Zareparsi et al., 2005; Conley et al., 2005; Schmidt et al., 2006).

However, humans have two genomes—nuclear and mitochondrial. The mitochondrial genome consists of only 16,569 base pairs. This small, circular genome encodes for vitally important subunits in the mitochondrial electron transport chain as well as a complete set of tRNAs and rRNAs (Wallace, 1999; DiMauro S, Schon, 2003; Wallace, 1994; Wallace et al., 1999). Stable single nucleotide polymorphisms (snps) have emerged in the mitochondrial genome over the past 150,000 years (Cann et al. 1984). Related combinations of these mtDNA polymorphisms are called haplogroups. The distribution of mitochondrial haplogroups differs between continents and populations reflecting both human migration and acquired genetic variation (Wallace et al., 1999; Anderson et al., 1981; Torroni et al., 1996; Herrnstadt and Howell, 2004). Changes in the mitochondrial genome have been associated with neurodegenerative disorders, including Parkinson disease, Alzheimer disease, Friedrich's ataxia, and amytrophic lateral sclerosis (van der Walt et al., 2003; van der Walt et al., 2004; Shoffner et al., 1993; Giacchetti et al., 2004; Mancuso et al., 2004).

Mitochondria are cytoplasmic organelles that play a central role in cellular energy production, free radical production, and apoptosis. Each of these processes has been considered in the pathogenesis of AMD (Ohia et al., 2005; Moriarty-Craige et al., 2005; Dunaief et al., 2002). Under normal physiologic conditions, electrons leak from the mitochondrial electron transport chain and reduce oxygen to superoxide anion, initiating a cascade of free radicals, called reactive oxygen species (ROS) that indiscriminately damage biological macromolecules (Beatty et al., 2000; Spraul et al., 1996). These free radicals damage the mitochondria and thus can compromise the production of ATP and trigger apoptosis. Previous research has demonstrated that retinal pigment epithelium is especially susceptible to damage by ROS (Ballinger et al., 1999; Jin et al., 2001; Alge et al., 2002; Liang et al., 2004). Antioxidants appear to ameliorate this effect (Liang et al., 2004). Mitochondria are especially susceptible to damage by these ROS because the mitochondrial genome lacks introns and has limited reparative capacity (Wallace et al., 1999). Susceptibility to this attack by ROS, and hence susceptibility to AMD, may differ depending on genetic variation within an individual's mitochondrial genome.

### SUMMARY OF THE INVENTION

Thus, in accordance with the present invention, there is provided a method of screening an individual for susceptibility to age-related macular degeneration (AMD) comprising assessing (a) the individual's structure and/or function of a Complement factor H(CFH) protein; (b) the individual's structure at LOC387715/ARMS2; and (c) the interaction between the individual's structure at LOC387715/ARMS2 and the individual's smoking history. One may also consider variation in the C3 and/or C2/CFB and/or mitochondrial nucleic acid sequence genes or gene products. The AMD may be wet AMD or dry AMD.

Step (a) may comprise assessing the structure of a CFH-encoding nucleic acid from said individual, determining the CFH-encoding nucleic acid sequence for all or a portion of a CFH-encoding RNA, including but not restricted to the CFH-encoding nucleic acid sequence corresponding to position Y402. This may also comprise sequencing or differential hybridization, and may also comprise amplifying all or part of said individual's CFH-encoding nucleic acid, such as by polymerase chain reaction (PCR). Determining the sequence CFH-encoding nucleic acid sequence may also comprise determining the sequence of a nucleic acid that is in linkage disequilibrium with position Y402. Step (a) may alternatively comprise assessing the function of a CFH protein from said individual.

Step (b) may comprise assessing the presence or absence of a T allele at rs10490924, such as by sequencing or differential hybridization. This may also comprise amplifying all or part of said individual's nucleic acid at rs10490924, such as by PCR. Assessing may also comprise determining the sequence of a nucleic acid that is in linkage disequilibrium with rs10490924. Assessing may even comprise the expression or activity of the gene located at LOC387715/ARMS2.

Step (c) may comprise assessing a person's smoking history through direct or indirect questioning, and statistically combining this information with the results from step (b). This may comprise determining a product of the codes for individual susceptibility factors.

The method may further comprise assessing genetic variation in said individual's mtDNA. Assessing mtDNA may comprise determining the mtDNA sequence at positions corresponding to the MTND1*LHON4216C and/or MTND2*LHON4917G alleles, such as by sequencing of all or a portion of a corresponding RNA. This may also comprise amplifying all or part of said individual's mtDNA, such as by PCR. Determining the sequence of said individual's mtDNA may involve determining the sequence of a mitochondrial RNA that comprises a polymorphism that is in linkage disequilibrium with the MTND1*LHON4216C and/or MTND2*LHON4917G alleles. This too may comprise amplifying all or part of said individual's mtDNA, such as by PCR. Alternatively, assessing the presence or absence of the MTND1*LHON4216C and/or MTND2*LHON4917G alleles comprises assessing the presence or absence of the MTND1*LHON4216C and/or MTND2*LHON4917G alleles in said individual's maternal blood relative's mtDNA. Assessing the presence or absence of the MTND1*LHON4216C and/or MTND2*LHON4917G alleles may comprise differential hybridization.

The method may also further comprise: (i) assessing genetic variation in said individual's C2/CFB gene cluster; (ii) assessing genetic variation in said individual's C3 gene locus; and/or (iii) assessing genetic variation in said individual's VEGF gene locus.

In another embodiment, there is provided a method for determining the need for prophylactic treatment for age-related macular degeneration (AMD) comprising assessing (a) the individual's structure and/or function of a Complement factor H (CHF) protein; (b) the individual's structure at LOC387715/ARMS2; and (c) the interaction between the individual's structure at LOC387715/ARMS2 and the individual's smoking history. The method may further comprise assessing genetic variation in said individual's mtDNA, and/or may further comprise assessing genetic variation in said individual's C2/CFB gene cluster, genetic variation in said individual's C3 gene locus, and/or genetic variation in said individual's VEGF gene locus.

In a further embodiment, there is provided a kit comprising, in a suitable container means, at least one nucleic acid for determining (i) the presence or absence of one or more of (a) a T allele at rs10490924 or (b) a variation in the Complement H gene; and (ii) the structure at LOC387715/ARMS2. The kit may further comprise (iii) a nucleic acid for determining the presence or absence of the MTND1*LHON4216C allele and/or at least one nucleic acid for determining the presence or absence of the MTND2*LHON4917G allele; (iv) a nucleic acid for determining a sequence with a C3 gene locus; (v) a nucleic acid for determining a sequence with a C2/CFB gene cluster; and/or (vi) a nucleic acid for determining a sequence within an VEGF gene locus.

It is contemplated that any method or composition described herein can be implemented with respect to any other method or composition described herein. Similarly, any embodiment discussed with respect to one aspect of the invention may be used in the context of any other aspect of the invention.

Throughout this application, the term “about” is used to indicate that a value includes the standard deviation of error for the device or method being employed to determine the value.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawing forms part of the present specification and is included to further demonstrate certain aspects of the present invention. The invention may be better understood by reference to the drawing in combination with the detailed description of specific embodiments presented herein.

FIG. 1—Overview of the Model for Predicting AMD Status.

FIG. 2—Training Dataset Used to Build of Decision Tree Rules. Note—these rules may change depending on what specific factors are including in the model, and can easily be added to as new susceptibility factors for AMD are discovered.

### DETAILED DESCRIPTION OF ILLUSTRATIVE EMBODIMENTS

Age-related macular degeneration (AMD) is the most common cause of irreversible vision loss in the developed world (Tielsch et al., 1995; Klayer et al., 1998; Attebo et al., 1996). In most patients, the disease is manifest as ophthalmoscopically visible yellowish accumulations of protein and lipid (known as drusen) that lie beneath the retinal pigment epithelium (RPE) and within a multi-layered structure known as Bruch's membrane. The central layer of Bruch's membrane is composed largely of elastin, and this layer is sandwiched between two collagenous sheets. The basal laminae of the RPE (on the retinal side) and the choriocapillaris (on the choroidal side) lie upon these sheets of collagen to complete the five layered structure. In approximately 10% of AMD patients, the disease is further complicated by the abnormal growth of new blood vessels from the choriocapillaris, through Bruch's membrane and into the sub-RPE or subretinal space (Ferris et al., 1984).

The clinical entity known as AMD is likely to be a mechanistically heterogeneous group of disorders. At this time, the specific disease mechanisms that underlie the vast majority of cases of age related macular degeneration are unknown. However, a number of studies have suggested that both genetic and environmental factors are likely to play a role in most patients (Heiba et al., 1994; Seddon et al., 1997; Klayer et al., 1998). Several investigators have used a population-based epidemiologic approach to try to identify specific environmental insults that might increase an individual's risk for AMD (Smith et al., 2001; Seddon et al., 1994). These studies have revealed some factors that appear to modify or exacerbate the disease (smoking is the most significant of the latter) (Smith et al., 2001), but none that are likely to be causative. This is perhaps understandable given the high prevalence, late onset, and slow progression of the disease. On the genetic side, AMD is equally challenging. Based on the study of other inherited retinal disorders, AMD is likely to display extensive genetic heterogeneity, involving functional sequence variations in numerous genes, sometimes singly, and sometimes in combination. Given the fact that AMD takes six decades or more to become clinically manifest in most patients, many of these variations are likely to have subtle effects on the proteins they encode and will therefore display variable expressivity and incomplete penetrance.

Despite these challenges, there are several advantages to probing the complex pathogenesis of AMD with genetic methods. First, the techniques and genomic data developed during the Human Genome Project make it easier to reliably screen selected portions of the genomes of elderly patients than to query their environmental exposures. Second, one can often use knowledge of phenotype-altering genetic variations in humans to create animal or in vitro models of these diseases. Such models would be of value to the pharmaceutical industry in their search for small molecule drugs that are capable of mitigating one or more AMD phenotypes. Finally, once such drugs are developed, one could use genetic data from the human population to identify patients who might benefit from treatment prior to the onset of symptoms or signs, thereby allowing physicians to prevent or delay the development of the disease.

In the past decade, many groups used positional cloning to try to identify genes that cause early-onset heritable macular diseases in the hope that identification of these genes would provide insight into the late-onset forms of this disease. Several genes were identified with this approach (Allikmets et al., 1997; Petrukhin et al., 1998; Weber et al., 1994; Nichols et al., 1993; Zhang et al., 2001; Stone et al., 1999) but none of them have been convincingly demonstrated to be involved in a significant fraction of late-onset macular degeneration (Stone et al., 1998; Lotery et al., 2000). Recently, a strong association between AMD and a coding variant (Y402H) in the complement factor H gene (CFH) was identified (odds ratios between 2.45 and 5.57) (PCT Application PCTUS06/007). This same study also implicated a coding change in LOC387715 (rs10490924) as a second major AMD susceptibility allele, albeit independent of the CFH variation. The joint effect of these two susceptibility genes is consistent with a multiplicative model, and together, they may explain as much as 65% of the PAR of AMD. Moreover, the effect of the rs10490924 variation is strongly modified by cigarette smoking, potentially explaining as much as 34% of AMD. Additional reports have implicated variations in additional genes including C3, C2/CFB, and VEGF.

Subsequently, the present inventors observed that study participants with specific mitochondrial DNA polymorphisms that alter the amino acid sequence of important subunits in the electron transport chain have an increased risk for AMD. This discovery adds to the growing evidence that mitochondrial genetic variation may play an important role in neurodegenerative processes (van der Walt et al., 2003; van der Walt et al., 2004; Shoffner et al., 1993; Giacchetti et al., 2004; Mancuso et al., 2004; Hulgan et al., 2005). These observations are consistent with the fact that since mitochondria are vitally important in free radical production, apoptosis and cellular energy production. However, the fact that these cytoplasmic organelles have their own genome and that variation in this distinct maternally inherited DNA may impact on neurodegeneration has been underappreciated. While this study provided evidence of a clear relationship between these two mitochondrial polymorphisms and susceptibility to developing AMD, a more sophisticated approach to the examination of AMD was required.

In summary, the studies reported here provide the first evidence that variation in the mitochondrial genome contributes to susceptibility to AMD. The magnitude of the risk associated with the MTND1*LHON4216C allele and particularly the MTND2*LHON4917G allele suggest that they constitute important new risk factors for the development of AMD. These markers can further be used in conjunction with environmental/behavioral risk factors, as well as any previously identified genetic markers.

### I. Age-Related Macular Degeneration

Macular degeneration is the leading cause of blindness in individuals over 55. It is caused by the physical disturbance of the center of the retina, called the macula. The macula is the part of the retina which is responsible for the most acute and detailed vision. Therefore, it is critical for reading, driving, recognizing faces, watching television, and fine work. Even with a loss of central vision, however, color vision and peripheral vision may remain clear. Vision loss usually occurs gradually and typically affects both eyes at different rates.

The root causes of macular degeneration are still unknown. There are two forms of age-related macular degeneration, “wet” and “dry.” Approximately seventy percent of patients have the dry form, which involves thinning of the macular tissues and disturbances in its pigmentation. Approximately thirty percent have the wet form, which can involve bleeding within and beneath the retina, opaque deposits, and eventually scar tissue. The wet form accounts for ninety percent of all cases of legal blindness in macular degeneration patients. Different forms of macular degeneration may occur in younger patients. These non-age related cases may be linked to heredity, diabetes, nutritional deficits, head injury, infection, or other factors.

Declining vision noticed by the patient or by an ophthalmologist during a routine eye exam may be the first indicator of macular degeneration. The formation of new blood vessels and exudates, or “drusen,” from blood vessels in and under the macular is often the first physical sign that macular degeneration may develop. In addition, the following signs may be indicative of macular problems. Other symptoms indicative of developing macular degeneration include (a) straight lines appear distorted and, in some cases, the center of vision appears more distorted than the rest of the scene; (b) a dark, blurry area or “white-out” appears in the center of vision; (c) color perception changes or diminishes. In the early stages, only one eye may be affected, but as the disease progresses, both eyes are usually affected.

Early detection is important because a patient destined to develop macular degeneration can sometimes be treated before symptoms appear, and this may delay or reduce the severity of the disease. Furthermore, as better treatments for macular degeneration are developed, whether medicinal, surgical, or low vision aids, patients diagnosed with macular degeneration can sooner benefit from them. However, there presently is no cure for macular degeneration. In some cases, macular degeneration may be active and then slow down considerably, or even stop progressing for many, many years. As discussed below, there are ways to slow or arrest macular degeneration, depending on the type and the degree of the condition.

### II. The Model

The present invention relies on the assembly of several prior risk factors reported for AMD, the combination being reliant to some extent on the interaction of certain factors. These factors and interactions are set forth below.

1. CFH

As discussed above, multiple independent research efforts identified the Y402H variant in the complement factor H (CFH [(MIM 134370]) gene on chromosome 1q32 as the first major AMD susceptibility allele (Haines et al., 2005; Hageman et al., 2005; Klein et al., 2005; Edwards et al., 2005; Zareparsi et al., 2005; Conley et al., 2005). While one of the studies was able to pinpoint CFH on the basis of a whole-genome association study (Klein et al., 2005), most studies focused on the 1q32 region because it had consistently been implicated by several whole-genome linkage scans. A second genomic region with similarly consistent linkage evidence is chromosome 10q26, which was identified as the single most promising region by a recent meta-analysis of published linkage screens (Fisher et al., 2005). In an earlier PCT application, PCTUS/06/007, incorporated herein by reference, the use of this marker to identify patients at risk of AMD is described.

2. LOC387715 and Smoking

Two recent studies have suggested specific AMD susceptibility genes located on chromosome 10q26. One used a combination of family-based and case-control analyses to implicate the PLEKHA1 gene (pleckstrin homology domain containing, family A (phosphoinositide binding specific) member 1 [MIM 607772]) and the predicted LOC387715 gene (Jakobsdottir et al., 2005). However, the association signals for single-nucleotide polymorphisms (SNPs) in these two genes were statistically indistinguishable. A second study using two independent case-control datasets concluded that the T allele of SNP rs10490924 in LOC387715, a coding change (Ala69Ser) in exon 1 of this poorly characterized gene, was the most likely AMD susceptibility allele (Rivera et al., 2005). Both studies reported that the chromosome 10q26 variant confers an AMD risk similar in magnitude to that of the Y402H variant in CFH.

PCT Application PCTUS/06/007, incorporated herein by reference, highly significant association of SNPs in LOC387715 with AMD was reported. The data from that application show that only SNPs in this gene, including rs10490924, explain the strong linkage and association signal in this region. Given a previous report of an effect of cigarette smoking on the linkage evidence in the 10q26 region (Weeks et al., 2004), the inventors then tested whether smoking modified this association, and found significantly increased risk associated with this behavior. The risk related to inheritance of the T allele at rs10490924, in the absence of a smoking history in that person, explains only a small proportion of the overall risk related to either smoking or LOC387715 alone. The primary risk arises from the combination of the T allele at rs10490924 and a history of smoking.

3. Mitochondrial Sequences

Prior epidemiologic evidence in AMD pointed to a preponderance of cases among individual of European heritage (The Eye Diseases Prevalence Research Group., 2004; Sommer et al., 1991). In the inventors' initial study, reported here, the inventors determined European mitochondrial haplogroups in series of AMD cases and controls and found that a specific combination of related European haplogroups, haplogroups J and T, appeared to have a significantly increased risk for AMD. This pattern was similar to the distribution of mitochondrial haplogroups recently seen in HIV patients developing peripheral neuropathy on nucleoside reverse transcriptase inhibitors, drugs that have been previously implicated in mitochondrial injury (Hulgan et al., 2005; Browne et al., 1993; Kelleher et al., 1999; Cui et al., 1997; Dalakas et al., 2001). The J and T haplogroups share a snp, MTND1*LHON4216C, that has been previously associated with Leber's Hereditary Optic Neuropathy (LHON) (Torroni et al., 1997; Hofmann et al., 1997; Howell et al., 1995; Fauser et al., 2002).

It was indeed serendipitous that mitochondrial haplogroups J, and particularly, T appear to confer increased risk for the development of AMD. The inventors recently saw the same pattern of haplogroup distribution while studying human immunodeficiency virus (HIV) patients who developed peripheral neuropathy while on mitochondrially-toxic antiretroviral therapy (Hulgan et al., 2005). Among Caucasian HIV patients in that study, the haplgroup T had an OR=2.8 (95% CI 1.1-7.1, p=0.03 (Hulgan et al., 2005). The primary single nucleotide polymorphism (snp) that distinguishes haplogroup T from other Caucasian haplogroups is located at position 13368 in the mitochondrial genome and is synonymous, that is it does not result in a change in the amino acid sequence (Torroni et al., 1996). Therefore, it is unlikely that this particular snp resulted in the alteration of mitochondrial function that could account for the either the AMD phenotype or the peripheral neuropathy. The inventors searched for other polymorphisms that could be shared by both J and T as well as potentially functional polymorphisms confined to haplogroup T. Ruiz-Pessini et al discovered that haplogroup T was significantly more common in the population of infertile men they studied (Ruiz-Pesini et al., 2000). These men with haplogroup T mitochondria had decreased sperm motility as well as diminished Complex I activity (Ruiz-Pesini et al., 2000). Haplogroup T also was subsequently found to be associated with increased susceptibility to Parkinson disease as well as decreased longevity (Ross et al., 2003; Ross et al., 2001). Both haplogroups J and T share a moderately conserved polymorphism at position 4216, MTND1*LHON4126C. This particular polymorphism has been previously associated with another retinal phenotype, Leber's Hereditary Optic Neuropathy (Brown et al., 1997; Torroni et al., 1997). Interestingly, haplogroup J has the polymorphism at position 10398 that has been protective in the susceptibility to Parkinson disease (van der Walt et al., 2003). In this study, the J haplogroup has a lower risk of AMD than haplogroup T. This possibly could represent the effect of having the protective 10398 allele and the deleterious MTND1*LHON4126C allele. Haplogroup T does not have the protective allele. Haplogroup T also harbors an even more conserved snp at position 4917, MTND2*LHON4917G, that also results in a base pair change in ND2, a different subunit of Complex I of the mitochondrial electron transport chain. These finding should point the way for future studies in mitochondrial cybrids to measure the consequences these polymorphisms have on free radical generation as well as energy production.

MTND1*LHON4126C, in the modern nomenclature for mitochondrial polymorphisms, represents the ND1 location of the mitochondrial gene where the change is located, its LHON association, the numerical base-pair position (4216) and finally the nucleotide change to C from the more common T (Wallace et al., 1999). LHON is a maternally inherited form of progressive blindness with a rather acute onset associated with several well described mitochondrial mutations (Man et al., 2002). MTND1*LHON4126C is not among the three so-called primary LHON mutations. However, the variable penetrance of these primary mutations let to the discovery other mtDNA polymorphisms, including MTND1*LHON4126C, associated with LHON (Torroni et al., 1997; Hofmann et al., 1997; Howell et al., 1995; Fauser et al., 2002). These polymorphisms have been referred to as “secondary” LHON variants (Wallace et al., 1999). While acknowledging that these “secondary” polymorphisms seem to account for susceptibility to some neurodegenerative phenotypes, dropping the link with LHON has been advocated because these mutations are not truly pathognomic for LHON (Hoffman et al., 1997). However, the current nomenclature has yet to catch up to this idea. However, epidemiologic evidence continues to mount that supports the association of MTND1*LHON4126C with neurodegenerative phenotypes including Parkinson disease and also decreased longevity (Ross et al., 2003; Kirchner et al., 2000; Ross et al., 20010.

Within mitochondrial haplogroup T, there is even more highly conserved non-synonymous mtDNA polymorphism that has also been associated with LHON, MTND2*LHON4917G. This polymorphism is not found in haplogroup J. The inventors were presented with a special opportunity to sort out the roles these mtDNA polymorphisms may play in the development of another neurodegenerative disorder, AMD. Thus, certain embodiments of the present invention concern various nucleic acids, including amplification primers, oligonucleotide probes, and other nucleic acid elements involved in the analysis of mt DNA, particularly MTND1*LHON4126C and MTND2*LHON4917G.

4. C2/CFB

Complement factor B is a component of the alternative pathway of complement activation. CFB circulates in the blood as a single chain polypeptide. Upon activation of the alternative pathway, it is cleaved by complement factor D, yielding the non-catalytic chain Ba and the catalytic subunit Bb. The active subunit Bb is a serine protease which associates with C3b to form the alternative pathway C3 convertase. Bb is involved in the proliferation of preactivated B lymphocytes, while Ba inhibits their proliferation. This gene localizes to the major histocompatibility complex (MHC) class III region on chromosome 6. This cluster includes several genes involved in regulation of the immune reaction. The polyadenylation site of this gene is 421 bp from the 5′ end of the gene for complement component 2. The mRNA accession no. is NM_{—}001710.

5. C3

Soluble C3-convertase, also known as iC3Bb, catalyzes the proteolytic cleavage of C3 into C3a and C3b as part of the alternative complement system. C3a plays an important role in chemotaxis, though not as important a role as C5a. It is also an anaphylatoxin. C3b may bind to microbial cell surfaces within an organism's body. This can lead to the production of surface-bound C3 convertase and thus more C3b components. Also known as C3bBb, this convertase is similar to soluble C3-convertase except that it is membrane bound. Alternatively, bound C3b may aid in opsonization of the microbe by macrophages. Complement receptor 1 or CR1 on macrophages allows the engaging of C3b covered microbes. C3b is cleaved into C3c and C3d. The C3 mRNA accession no. is NM_{—}000064.

6. Application of the Model to a Subject

To predict the risk of developing AMD in a single individual, assessments are made of the variation in the above mentioned measures. Current methods of risk assessment rely on a single method of combining results. In this embodiment, a model is constructed that employs multiple methods of combining the results of these measures and develops a consensus risk estimate for the individual. In particular, in addition to including the individual effects of each variation, the inventors include measures of the non-linear combination of these variations (e.g., statistical interaction). The multiple methods may include two or more of Grammatical Evolution Neural Networks (GENN), logistic regression equations, decision trees, Bayesian classification and multifactor dimensionality reduction (MDR). In each approach, the specific combination of measures that best predict an individual's risk is determined. The resulting risk measures are adjudicated to determine the most accurate risk assessment.

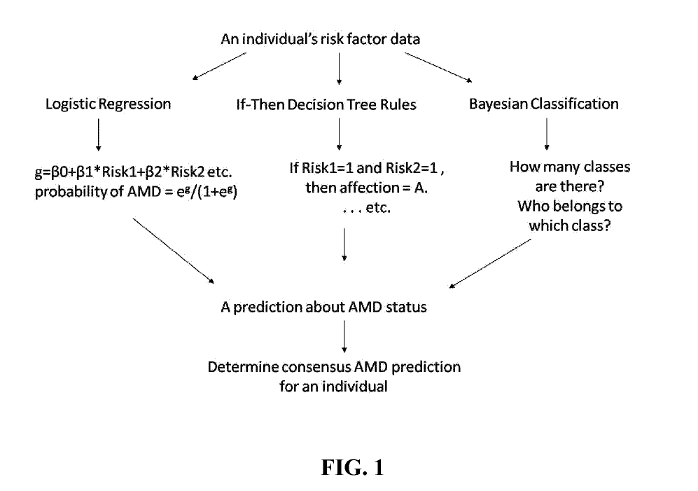

With the identification of both genetic and environmental risk factors for age-related macular degeneration, it is now possible to develop predictive models to assess an individual's chance of developing AMD based on their particular combination of susceptibility factors. Logistic regression (LR) analyses have been applied to this problem (Maller et al., 2006; Hughes et al., 2007), but so far this approach has failed: 1) to rigorously test the derived models on an independent dataset; 2) to include gene-environment interactions; and 3) to combine two or more approaches to arrive at a consensus prediction. The inventors' approach (FIG. 1) includes all 3 of these features, making it superior to previous versions.

### IV. Nucleic Acid Detection

Some embodiments of the invention concern identifying polymorphisms in sequences such a genomic DNA, mRNA and mtDNA, correlating to increased or decreased risk for developing AMD. Thus, the present invention involves assays for identifying polymorphisms and other nucleic acid detection methods. It is contemplated that probes and primers can be prepared based on previously published sequences for each of the targets. Nucleic acids, therefore, have utility as probes or primers for embodiments involving nucleic acid hybridization. They may be used in diagnostic or screening methods of the present invention. General methods of nucleic acid detection methods are provided below, followed by specific examples employed for the identification of polymorphisms, including single nucleotide polymorphisms (SNPs).

1. Hybridization

The use of a probe or primer of between 7, 8, 9, 10, 11, 12, 13, 14, 15, 16, 17, 18, 19, 20, 21, 22, 23, 24, 25, 50, 60, 70, 80, 90, or 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting a specific polymorphism. It is generally appreciated that conditions can be rendered more stringent by the addition of increasing amounts of formamide. For example, under highly stringent conditions, hybridization to filter-bound DNA may be carried out in 0.5 M NaHPO₄, 7% sodium dodecyl sulfate (SDS), 1 mM EDTA at 65° C., and washing in 0.1×SSC/0.1% SDS at 68° C. (Ausubel et al., 1989).

Conditions may be rendered less stringent by increasing salt concentration and/or decreasing temperature. For example, a medium stringency condition could be provided by about 0.1 to 0.25M NaCl at temperatures of about 37° C. to about 55° C., while a low stringency condition could be provided by about 0.15M to about 0.9M salt, at temperatures ranging from about 20° C. to about 55° C. Under low stringent conditions, such as moderately stringent conditions the washing may be carried out for example in 0.2×SSC/0.1% SDS at 42° C. (Ausubel et al., 1989). Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples. In other aspects, a particular nuclease cleavage site may be present and detection of a particular nucleotide sequence can be determined by the presence or absence of nucleic acid cleavage.

In general, it is envisioned that the probes or primers described herein will be useful as reagents in solution hybridization, as in PCR, for detection of expression or genotype of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples with or without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to the sequence flanking the target site of interest, or variants thereof, and fragments thereof are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids that contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected, analyzed or quantified. In certain applications, the detection may be performed by visual means. In certain applications, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA) (described in further detail below), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, Great Britain Application 2 202 328, and in PCT Application PCT/US89/01025, each of which is incorporated herein by reference in its entirety. Qbeta Replicase, described in PCT Application PCT/US87/00880, may also be used as an amplification method in the present invention.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application 329 822 disclose a nucleic acid amplification process involving cyclically synthesizing single-stranded RNA (ssRNA), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (ssDNA) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by spin columns and/or chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized, with or without separation. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Other Assays

Other methods for genetic screening may be used within the scope of the present invention, for example, to detect mutations in genomic DNA, cDNA and/or RNA samples. Methods used to detect point mutations include denaturing gradient gel electrophoresis (DGGE), restriction fragment length polymorphism analysis (RFLP), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

5. Specific Examples of Polymorphism Screening Methods

Spontaneous mutations that arise during the course of evolution in the genomes of organisms are often not immediately transmitted throughout all of the members of the species, thereby creating polymorphic alleles that co-exist in the species populations. Often polymorphisms are the cause of genetic diseases. Several classes of polymorphisms have been identified. For example, variable nucleotide type polymorphisms (VNTRs), arise from spontaneous tandem duplications of di- or trinucleotide repeated motifs of nucleotides. If such variations alter the lengths of DNA fragments generated by restriction endonuclease cleavage, the variations are referred to as restriction fragment length polymorphisms (RFLPs). RFLPs have been widely used in human and animal genetic analyses.

Another class of polymorphisms are generated by the replacement of a single nucleotide. Such single nucleotide polymorphisms (SNPs) rarely result in changes in a restriction endonuclease site. Thus, SNPs are rarely detectable restriction fragment length analysis. SNPs are the most common genetic variations and occur once every 100 to 300 bases and several SNP mutations have been found that affect a single nucleotide in a protein-encoding gene in a manner sufficient to actually cause a genetic disease. SNP diseases are exemplified by hemophilia, sickle-cell anemia, hereditary hemochromatosis, late-onset alzheimer disease etc.

SNPs can be the result of deletions, point mutations and insertions and in general any single base alteration, whatever the cause, can result in a SNP. The greater frequency of SNPs means that they can be more readily identified than the other classes of polymorphisms. The greater uniformity of their distribution permits the identification of SNPs “nearer” to a particular trait of interest. The combined effect of these two attributes makes SNPs extremely valuable. For example, if a particular trait (e.g., inability to efficiently metabolize irinotecan) reflects a mutation at a particular locus, then any polymorphism that is linked to the particular locus can be used to predict the probability that an individual will be exhibit that trait.

Several methods have been developed to screen polymorphisms and some examples are listed below. The reference of Kwok and Chen (2003) and Kwok (2001) provide overviews of some of these methods; both of these references are specifically incorporated by reference.

SNPs or other polymorphisms relating to mtDNA position 10398 can be characterized by the use of any of these methods or suitable modification thereof. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or any other biochemical interpretation.

a. DNA Sequencing

The most commonly used method of characterizing a polymorphism is direct DNA sequencing of the genetic locus that flanks and includes the polymorphism. Such analysis can be accomplished using either the “dideoxy-mediated chain termination method,” also known as the “Sanger Method” (Sanger et al., 1975) or the “chemical degradation method,” also known as the “Maxam-Gilbert method” (Maxam et al., 1977). Sequencing in combination with genomic sequence-specific amplification technologies, such as the polymerase chain reaction may be utilized to facilitate the recovery of the desired genes (Mullis et al., 1986; European Patent Application 50,424; European Patent Application. 84,796, European Patent Application 258,017, European Patent Application. 237,362; European Patent Application. 201,184; U.S. Pat. Nos. 4,683,202; 4,582,788; and 4,683,194), all of the above incorporated herein by reference.

b. Exonuclease Resistance

Other methods that can be employed to determine the identity of a nucleotide present at a polymorphic site utilize a specialized exonuclease-resistant nucleotide derivative (U.S. Pat. No. 4,656,127). A primer complementary to an allelic sequence immediately 3′-to the polymorphic site is hybridized to the DNA under investigation. If the polymorphic site on the DNA contains a nucleotide that is complementary to the particular exonucleotide-resistant nucleotide derivative present, then that derivative will be incorporated by a polymerase onto the end of the hybridized primer. Such incorporation makes the primer resistant to exonuclease cleavage and thereby permits its detection. As the identity of the exonucleotide-resistant derivative is known one can determine the specific nucleotide present in the polymorphic site of the DNA.

c. Microsequencing Methods

Several other primer-guided nucleotide incorporation procedures for assaying polymorphic sites in DNA have been described (Komher et al., 1989; Sokolov, 1990; Syvanen 1990; Kuppuswamy et al., 1991; Prezant et al., 1992; Ugozzoll et al., 1992; Nyren et al., 1993). These methods rely on the incorporation of labeled deoxynucleotides to discriminate between bases at a polymorphic site. As the signal is proportional to the number of deoxynucleotides incorporated, polymorphisms that occur in runs of the same nucleotide result in a signal that is proportional to the length of the run (Syvanen et al., 1990).

d. Extension in Solution

French Patent 2,650,840 and PCT Application WO91/02087 discuss a solution-based method for determining the identity of the nucleotide of a polymorphic site. According to these methods, a primer complementary to allelic sequences immediately 3′-to a polymorphic site is used. The identity of the nucleotide of that site is determined using labeled dideoxynucleotide derivatives which are incorporated at the end of the primer if complementary to the nucleotide of the polymorphic site.

e. Genetic Bit Analysis or Solid-Phase Extension

PCT Application WO92/15712 describes a method that uses mixtures of labeled terminators and a primer that is complementary to the sequence 3′ to a polymorphic site. The labeled terminator that is incorporated is complementary to the nucleotide present in the polymorphic site of the target molecule being evaluated and is thus identified. Here the primer or the target molecule is immobilized to a solid phase.

f. Oligonucleotide Ligation Assay (OLA)

This is another solid phase method that uses different methodology (Landegren et al., 1988). Two oligonucleotides, capable of hybridizing to abutting sequences of a single strand of a target DNA are used. One of these oligonucleotides is biotinylated while the other is detectably labeled. If the precise complementary sequence is found in a target molecule, the oligonucleotides will hybridize such that their termini abut, and create a ligation substrate. Ligation permits the recovery of the labeled oligonucleotide by using avidin. Other nucleic acid detection assays, based on this method, combined with PCR have also been described (Nickerson et al., 1990). Here PCR is used to achieve the exponential amplification of target DNA, which is then detected using the OLA.

g. Ligase/Polymerase-Mediated Genetic Bit Analysis

U.S. Pat. No. 5,952,174 describes a method that also involves two primers capable of hybridizing to abutting sequences of a target molecule. The hybridized product is formed on a solid support to which the target is immobilized. Here the hybridization occurs such that the primers are separated from one another by a space of a single nucleotide. Incubating this hybridized product in the presence of a polymerase, a ligase, and a nucleoside triphosphate mixture containing at least one deoxynucleoside triphosphate allows the ligation of any pair of abutting hybridized oligonucleotides. Addition of a ligase results in two events required to generate a signal, extension and ligation. This provides a higher specificity and lower “noise” than methods using either extension or ligation alone and unlike the polymerase-based assays, this method enhances the specificity of the polymerase step by combining it with a second hybridization and a ligation step for a signal to be attached to the solid phase.

h. Invasive Cleavage Reactions

Invasive cleavage reactions can be used to evaluate cellular DNA for a particular polymorphism. A technology called INVADER® employs such reactions (e.g., de Arruda et al., 2002; Stevens et al., 2003, which are incorporated by reference). Generally, there are three nucleic acid molecules: 1) an oligonucleotide upstream of the target site (“upstream oligo”), 2) a probe oligonucleotide covering the target site (“probe”), and 3) a single-stranded DNA with the target site (“target”). The upstream oligo and probe do not overlap but they contain contiguous sequences. The probe contains a donor fluorophore, such as fluoroscein, and an acceptor dye, such as Dabcyl. The nucleotide at the 3′ terminal end of the upstream oligo overlaps (“invades”) the first base pair of a probe-target duplex. Then the probe is cleaved by a structure-specific 5′ nuclease causing separation of the fluorophore/quencher pair, which increases the amount of fluorescence that can be detected. See Lu et al. (2004). In some cases, the assay is conducted on a solid-surface or in an array format.

i. Other Methods to Detect SNPs

Several other specific methods for SNP detection and identification are presented below and may be used as such or with suitable modifications in conjunction with identifying polymorphisms (directly or indirectly) at mtDNA position 10398. Several other methods are also described on the SNP web site of the NCBI at the website on the World Wide Web at ncbi.nlm.nih.gov/SNP, incorporated herein by reference.

In a particular embodiment, extended sequence information may be determined at any given locus in a population, which allows one to identify exactly which SNPs will be redundant and which will be essential in association studies. In studies of genomic DNA material the latter is referred to as ‘haplotype tag SNPs (htSNPs),’ markers that capture the haplotypes of a gene or a region of linkage disequilibrium. See Johnson et al. (2001) and Ke and Cardon (2003), each of which is incorporated herein by reference, for exemplary methods.

The VDA-assay utilizes PCR amplification of genomic segments by long PCR methods using TaKaRa LA Taq reagents and other standard reaction conditions. The long amplification can amplify DNA sizes of about 2,000-12,000 bp. Hybridization of products to variant detector array (VDA) can be performed by a Affymetrix High Throughput Screening Center and analyzed with computerized software.

A method called Chip Assay uses PCR amplification of genomic segments by standard or long PCR protocols. Hybridization products are analyzed by VDA, Halushka et al. (1999), incorporated herein by reference. SNPs are generally classified as “Certain” or “Likely” based on computer analysis of hybridization patterns. By comparison to alternative detection methods such as nucleotide sequencing, “Certain” SNPs have been confirmed 100% of the time; and “Likely” SNPs have been confirmed 73% of the time by this method.

Other methods simply involve PCR amplification following digestion with the relevant restriction enzyme. Yet others involve sequencing of purified PCR products from known genomic regions.

In yet another method, individual exons or overlapping fragments of large exons are PCR-amplified. Primers are designed from published or database sequences and PCR-amplification of genomic DNA is performed using the following conditions: 200 ng DNA template, 0.5 μM each primer, 80 μM each of dCTP, dATP, dTTP and dGTP, 5% formamide, 1.5 mM MgCl₂, 0.5 U of Taq polymerase and 0.1 volume of the Taq buffer. Thermal cycling is performed and resulting PCR-products are analyzed by PCR-single strand conformation polymorphism (PCR-SSCP) analysis, under a variety of conditions, e.g, 5 or 10% polyacrylamide gel with 15% urea, with or without 5% glycerol. Electrophoresis is performed overnight. PCR-products that show mobility shifts are reamplified and sequenced to identify nucleotide variation.

In a method called CGAP-GAI (DEMIGLACE), sequence and alignment data (from a PHRAP.ace file), quality scores for the sequence base calls (from PHRED quality files), distance information (from PHYLIP dnadist and neighbour programs) and base-calling data (from PHRED ‘-d’ switch) are loaded into memory. Sequences are aligned and examined for each vertical chunk (‘slice’) of the resulting assembly for disagreement. Any such slice is considered a candidate SNP (DEMIGLACE). A number of filters are used by DEMIGLACE to eliminate slices that are not likely to represent true polymorphisms. These include filters that: (i) exclude sequences in any given slice from SNP consideration where neighboring sequence quality scores drop 40% or more; (ii) exclude calls in which peak amplitude is below the fifteenth percentile of all base calls for that nucleotide type; (iii) disqualify regions of a sequence having a high number of disagreements with the consensus from participating in SNP calculations; (iv) removed from consideration any base call with an alternative call in which the peak takes up 25% or more of the area of the called peak; (v) exclude variations that occur in only one read direction. PHRED quality scores were converted into probability-of-error values for each nucleotide in the slice. Standard Baysian methods are used to calculate the posterior probability that there is evidence of nucleotide heterogeneity at a given location.

In a method called CU-RDF (RESEQ), PCR amplification is performed from DNA isolated from blood using specific primers for each SNP, and after typical cleanup protocols to remove unused primers and free nucleotides, direct sequencing using the same or nested primers.

In a method called DEBNICK (METHOD-B), a comparative analysis of clustered EST sequences is performed and confirmed by fluorescent-based DNA sequencing. In a related method, called DEBNICK (METHOD-C), comparative analysis of clustered EST sequences with phred quality >20 at the site of the mismatch, average phred quality >=20 over 5 bases 5′-FLANK and 3′ to the SNP, no mismatches in 5 bases 5′ and 3′ to the SNP, at least two occurrences of each allele is performed and confirmed by examining traces.

In a method identified by ERO (RESEQ), new primers sets are designed for electronically published STSs and used to amplify DNA from 10 different mouse strains. The amplification product from each strain is then gel purified and sequenced using a standard dideoxy, cycle sequencing technique with ³³P-labeled terminators. All the ddATP terminated reactions are then loaded in adjacent lanes of a sequencing gel followed by all of the ddGTP reactions and so on. SNPs are identified by visually scanning the radiographs.

In another method identified as ERO (RESEQ-HT), new primers sets are designed for electronically published murine DNA sequences and used to amplify DNA from 10 different mouse strains. The amplification product from each strain is prepared for sequencing by treating with Exonuclease I and Shrimp Alkaline Phosphatase. Sequencing is performed using ABI Prism Big Dye Terminator Ready Reaction Kit (Perkin-Elmer) and sequence samples are run on the 3700 DNA Analyzer (96 Capillary Sequencer).

FGU-CBT (SCA2-SNP) identifies a method where the region containing the SNP were PCR amplified using the primers SCA2-FP3 and SCA2-RP3. Approximately 100 ng of genomic DNA is amplified in a 50 ml reaction volume containing a final concentration of 5 mM Tris, 25 mM KCl, 0.75 mM MgCl₂, 0.05% gelatin, 20 pmol of each primer and 0.5 U of Taq DNA polymerase. Samples are denatured, annealed and extended and the PCR product is purified from a band cut out of the agarose gel using, for example, the QIAquick gel extraction kit (Qiagen) and is sequenced using dye terminator chemistry on an ABI Prism 377 automated DNA sequencer with the PCR primers.

In a method identified as JBLACK (SEQ/RESTRICT), two independent PCR reactions are performed with genomic DNA. Products from the first reaction are analyzed by sequencing, indicating a unique FspI restriction site. The mutation is confirmed in the product of the second PCR reaction by digesting with Fsp I.

In a method described as KWOK(1), SNPs are identified by comparing high quality genomic sequence data from four randomly chosen individuals by direct DNA sequencing of PCR products with dye-terminator chemistry (see Kwok et al., 2003). In a related method identified as KWOK(2) SNPs are identified by comparing high quality genomic sequence data from overlapping large-insert clones such as bacterial artificial chromosomes (BACs) or P1-based artificial chromosomes (PACs). An STS containing this SNP is then developed and the existence of the SNP in various populations is confirmed by pooled DNA sequencing (see Taillon-Miller et al., 1998). In another similar method called KWOK(3), SNPs are identified by comparing high quality genomic sequence data from overlapping large-insert clones BACs or PACs. The SNPs found by this approach represent DNA sequence variations between the two donor chromosomes but the allele frequencies in the general population have not yet been determined. In method KWOK(5), SNPs are identified by comparing high quality genomic sequence data from a homozygous DNA sample and one or more pooled DNA samples by direct DNA sequencing of PCR products with dye-terminator chemistry. The STSs used are developed from sequence data found in publicly available databases. Specifically, these STSs are amplified by PCR against a complete hydatidiform mole (CHM) that has been shown to be homozygous at all loci and a pool of DNA samples from 80 CEPH parents (see Kwok et al., 1994).

In another such method, KWOK (OverlapSnpDetectionWithPolyBayes), SNPs are discovered by automated computer analysis of overlapping regions of large-insert human genomic clone sequences. For data acquisition, clone sequences are obtained directly from large-scale sequencing centers. This is necessary because base quality sequences are not present/available through GenBank. Raw data processing involves analyzed of clone sequences and accompanying base quality information for consistency. Finished ('base perfect', error rate lower than 1 in 10,000 bp) sequences with no associated base quality sequences are assigned a uniform base quality value of 40 (1 in 10,000 bp error rate). Draft sequences without base quality values are rejected. Processed sequences are entered into a local database. A version of each sequence with known human repeats masked is also stored. Repeat masking is performed with the program “MASKERAID.” Overlap detection: Putative overlaps are detected with the program “WUBLAST.” Several filtering steps followed in order to eliminate false overlap detection results, i.e., similarities between a pair of clone sequences that arise due to sequence duplication as opposed to true overlap. Total length of overlap, overall percent similarity, number of sequence differences between nucleotides with high base quality value “high-quality mismatches.” Results are also compared to results of restriction fragment mapping of genomic clones at Washington University Genome Sequencing Center, finisher's reports on overlaps, and results of the sequence contig building effort at the NCBI. SNP detection: Overlapping pairs of clone sequence are analyzed for candidate SNP sites with the ‘POLYBAYES’ SNP detection software. Sequence differences between the pair of sequences are scored for the probability of representing true sequence variation as opposed to sequencing error. This process requires the presence of base quality values for both sequences. High-scoring candidates are extracted. The search is restricted to substitution-type single base pair variations. Confidence score of candidate SNP is computed by the POLYBAYES software.

In method identified by KWOK (TaqMan assay), the TaqMan assay is used to determine genotypes for numerous random individuals (e.g., 384). The techniques is designed to be used in the case of a diploid genome (i.e., nuclear genetic material) but may also be employed to analyze mtDNA sequences. In method identified by KYUGEN(Q1), DNA samples of indicated populations are pooled and analyzed by PLACE-SSCP. Peak heights of each allele in the pooled analysis are corrected by those in a heterozygote, and are subsequently used for calculation of allele frequencies. Allele frequencies higher than 10% are reliably quantified by this method. Allele frequency=0 (zero) means that the allele was found among individuals, but the corresponding peak is not seen in the examination of pool. Allele frequency=0-0.1 indicates that minor alleles are detected in the pool but the peaks are too low to reliably quantify.

In yet another method identified as KYUGEN, PCR products are post-labeled with fluorescent dyes and analyzed by an automated capillary electrophoresis system under SSCP conditions (PLACE-SSCP). Four or more individual DNAs are analyzed with or without two pooled DNA (Japanese pool and CEPH parents pool) in a series of experiments. Alleles are identified by visual inspection. Individual DNAs with different genotypes are sequenced and SNPs identified. Allele frequencies are estimated from peak heights in the pooled samples after correction of signal bias using peak heights in heterozygotes. For the PCR primers are tagged to have 5′-ATT or 5′-GTT at their ends for post-labeling of both strands. Samples of DNA (10 ng/μl) are amplified in reaction mixtures containing the buffer (10 mM Tris-HCl, pH 8.3 or 9.3, 50 mM KCl, 2.0 mM MgCl₂), 0.25 μM of each primer, 200 μM of each dNTP, and 0.025 units/μl of Taq DNA polymerase premixed with anti-Taq antibody. The two strands of PCR products are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. For the SSCP: an aliquot of fluorescently labeled PCR products and TAMRA-labeled internal markers are added to deionized formamide, and denatured. Electrophoresis is performed in a capillary using an ABI Prism 310 Genetic Analyzer. Genescan softwares (P-E Biosystems) are used for data collection and data processing. DNA of individuals (two to eleven) including those who showed different genotypes on SSCP are subjected for direct sequencing using big-dye terminator chemistry, on ABI Prism 310 sequencers. Multiple sequence trace files obtained from ABI Prism 310 are processed and aligned by Phred/Phrap and viewed using Consed viewer. SNPs are identified by PolyPhred software and visual inspection.

In yet another method identified as KYUGEN, individuals with different genotypes are searched by denaturing HPLC (DHPLC) or PLACE-SSCP (Inazuka et al., 1997) and their sequences are determined to identify SNPs. PCR is performed with primers tagged with 5′-ATT or 5′-GTT at their ends for post-labeling of both strands. DHPLC analysis is carried out using the WAVE DNA fragment analysis system (Transgenomic). PCR products are injected into DNASep column, and separated under the conditions determined using WAVEMaker program (Transgenomic). The two strands of PCR products that are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. SSCP followed by electrophoresis is performed in a capillary using an ABI Prism 310 Genetic Analyzer. Genescan softwares (P-E Biosystems). DNA of individuals including those who showed different genotypes on DHPLC or SSCP are subjected for direct sequencing using big-dye terminator chemistry, on ABI Prism 310 sequencer. Multiple sequence trace files obtained from ABI Prism 310 are processed and aligned by Phred/Phrap and viewed using Consed viewer. SNPs are identified by PolyPhred software and visual inspection. Trace chromatogram data of EST sequences in Unigene are processed with PHRED. To identify likely SNPs, single base mismatches are reported from multiple sequence alignments produced by the programs PHRAP, BRO and POA for each Unigene cluster. BRO corrected possible misreported EST orientations, while POA identified and analyzed non-linear alignment structures indicative of gene mixing/chimeras that might produce spurious SNPs. Bayesian inference is used to weigh evidence for true polymorphism versus sequencing error, misalignment or ambiguity, misclustering or chimeric EST sequences, assessing data such as raw chromatogram height, sharpness, overlap and spacing; sequencing error rates; context-sensitivity; cDNA library origin, etc.

In another method, overlapping human DNA sequences which contained putative insertion/deletion polymorphisms are identified through searches of public databases. PCR primers which flanked each polymorphic site are selected from the consensus sequences. Primers are used to amplify individual or pooled human genomic DNA. Resulting PCR products are resolved on a denaturing polyacrylamide gel and a PhosphorImager is used to estimate allele frequencies from DNA pools.

6. Mass Spectrometry

Another methods uses mass spectrometry to determine the time of flight of the different molecules containing different allelic variants (Sequenom MassArray). This approach, know as QGE, combines the advantages of microarrays and real-time PCR, allowing the expression levels of large numbers of genes to be accurately quantified. The new technology starts with competitive PCR, followed by mass spectroscopy to allow highly accurate measurement of an intrinsic physical property of a nucleic acid molecule: its mass. This approach can be used to study hundreds, and in some cases thousands, of genes in large numbers of samples

QGE method starts with competitive a PCR reaction that contains a defined amount of an internal control which calibrates the reaction. The QGE assay is designed such that the competitor oligonucleotide has an identical sequence to the gene-region of interest except for a single artificially introduced base change. The cDNA and the competitor are then amplified in the same reaction, thus subjecting them to the same conditions throughout the assay. Once the competitive PCR assay is completed, the cDNA and the competitor are assayed using a simple primer extension reaction in the presence of a mixture of ddNTPs and dNTPs. The extended primers are designed to have different masses so that the products from the cDNA and the competitor can be distinguished through mass spectroscopy. See the world-wide-web at sequenom.com.

7. Linkage Disequilibrium

Polymorphisms in linkage disequilibrium with the number of TA repeats may also be used with the methods of the present invention. “Linkage disequilibrium” (“LD” as used herein, though also referred to as “LED” in the art) refers to a situation where a particular combination of alleles (i.e., a variant form of a given gene) or polymorphisms at two loci appears more frequently than would be expected by chance. “Significant” as used in respect to linkage disequilibrium, as determined by one of skill in the art, is contemplated to be a statistical p or a value that may be 0.25 or 0.1 and may be 0.1, 0.05. 0.001, 0.00001 or less.

### V. Antibodies and Protein Detection Methods

1. Antibodies

In certain aspects of the invention, one or more antibodies may be produced to the expressed ORF encoded by a mt gene, for example, MTND1*LHON4126C and MTND2*LHON4917G, or other target such as CHP. These antibodies may be used in various diagnostic applications, described herein.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab)₂, single domain antibodies (DABs), Fv, scFv (single chain Fv), and the like. The techniques for preparing and using various antibody-based constructs and fragments are well known in the art. Means for preparing and characterizing antibodies are also well known in the art (see, e.g., Antibodies: A Laboratory Manual, Cold Spring Harbor Laboratory, 1988; incorporated herein by reference).

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

The methods for generating monoclonal antibodies (MAbs) generally begin along the same lines as those for preparing polyclonal antibodies. Briefly, a polyclonal antibody is prepared by immunizing an animal with a LEE or CEE composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. The choice of animal may be decided upon the ease of manipulation, costs or the desired amount of sera, as would be known to one of skill in the art.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, chemokines, cofactors, toxins, plasmodia, synthetic compositions or LEEs or CEEs encoding such adjuvants.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/Mead, NJ), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen including but not limited to subcutaneous, intramuscular, intradermal, intraepidermal, intravenous and intraperitoneal. The production of polyclonal antibodies may be monitored by sampling blood of the immunized animal at various points following immunization.

A second, booster dose (e.g., provided in an injection), may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen, generally as described above. The antigen may be mixed with adjuvant, such as Freund's complete or incomplete adjuvant. Booster administrations with the same antigen or DNA encoding the antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). cites). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al., (1977). The use of electrically induced fusion methods is also appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. In one embodiment, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies. In another example, LEEs or CEEs can be used to produce antigens in vitro with a cell free system. These can be used as targets for scanning single chain antibody libraries. This would enable many different antibodies to be identified very quickly without the use of animals.

Alternatively, monoclonal antibody fragments encompassed by the present invention can be synthesized using an automated peptide synthesizer, or by expression of full-length gene or of gene fragments in E. coli.

2. Antibody Conjugates

The present invention further provides antibodies to ORF transcribed messages and translated proteins, polypeptides and peptides, generally of the monoclonal type, that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging.”

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴-carbon, ⁵¹chromium, ³⁶-chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

3. Immunodetection Methods

In still further embodiments, the present invention concerns immunodetection methods for binding, purifying, removing, quantifying and/or otherwise generally detecting biological components such as ORF expressed message(s), protein(s), polypeptide(s) or peptide(s). Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle and Ben-Zeev, 1999; Gulbis and Galand, 1993; De Jager et al., 1993; and Nakamura et al., 1987, each incorporated herein by reference.

In general, the immunobinding methods include obtaining a sample suspected of containing ORF expressed message and/or protein, polypeptide and/or peptide, and contacting the sample with a first anti-ORF message and/or anti-ORF translated product antibody in accordance with the present invention, as the case may be, under conditions effective to allow the formation of immunocomplexes.

These methods include methods for purifying an ORF message, protein, polypeptide and/or peptide from organelle, cell, tissue or organism's samples. In these instances, the antibody removes the antigenic ORF message, protein, polypeptide and/or peptide component from a sample. The antibody will preferably be linked to a solid support, such as in the form of a column matrix, and the sample suspected of containing the ORF message, protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen, and contact the sample with an antibody against the ORF produced antigen, and then detect and quantify the amount of immune complexes formed under the specific conditions.

In terms of antigen detection, the biological sample analyzed may be any sample that is suspected of containing an antigen, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum, although tissue samples or extracts are preferred.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any ORF antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The ORF antigen antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

a. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, the anti-ORF message and/or anti-ORF translated product antibodies of the invention are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another anti-ORF message and/or anti-ORF translated product antibody that is linked to a detectable label. This type of ELISA is a simple “sandwich ELISA.” Detection may also be achieved by the addition of a second anti-ORF message and/or anti-ORF translated product antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with the anti-ORF message and/or anti-ORF translated product antibodies of the invention. After binding and/or washing to remove non-specifically bound immune complexes, the bound anti-ORF message and/or anti-ORF translated product antibodies are detected. Where the initial anti-ORF message and/or anti-ORF translated product antibodies are linked to a detectable label, the immune complexes may be detected directly. Again, the immune complexes may be detected using a second antibody that has binding affinity for the first anti-ORF message and/or anti-ORF translated product antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

b. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 ng of frozen pulverized tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

### VI. Therapy and Prophylaxis

1. Prevention

Smoking can increase the risk of AMD, and is believed to be the most significant modifiable risk factor for developing this disease. It is also reported that risk factors for AMD include high blood pressure, obesity, heavy alcohol use, frequent consumption of processed baked goods, and long-term exposure to the sun. There is some belief that consumption of fish, nuts or other foods high in DHA and EPA may protect people from developing AMD. Recent studies have shown that the consumption of foods rich in antioxidants may lower AMD risk, leading to a suggestion that antioxidant supplementation (e.g., selenium) can aid subjects at risk of AMD. Certain fruits and vegetables, which are rich in the carotenoids lutein and zeaxanthin, may be protective as well.

2. Current Treatments

The Age-Related Eye Disease Study (AREDS) determined that antioxidant supplementation can slow the progression of AMD. The AREDS formulation is an over-the-counter antioxidant supplement recommended for people who are at risk of developing more advanced forms of either dry or wet AMD. The AREDS formulation includes the antioxidants beta carotene, vitamin E, and vitamin C, as well as the nutrients zinc and copper.

The FDA has approved Lucentis™ (ranibizumab) for the treatment of wet AMD. Developed by Genentech, Lucentis™ has been shown to be effective in reducing the risk of losing vision from abnormal blood vessel growth under the retina. Ninety-five percent of people with wet AMD who received monthly injections of Lucentis™ experienced no significant loss in visual acuity, and moderate visual improvement was reported in 24.8% of participants treated with a 0.3 mg dose and 33.8% of participants treated with a 0.5 mg dose.

Avastatin®, a drug similar to Lucentis™, has been used off-label by some ophthalmologists to treat wet AMD. In a few small clinical studies of short duration (e.g., three months), Avastin® appears to be safe and effective.

The FDA has also approved Macugen® for the treatment of wet AMD. Macugen has shown to be effective in reducing the risk of vision loss in people with wet AMD by inhibiting the growth of abnormal blood vessels under the retina. Typically, Macugen® is administered every six weeks through an injection into the eye. In clinical studies, approximately 70% of patients treated experienced no significant vision loss.

Visudyne® (verteporfin) photodynamic therapy (PDT) involves the use of a light-activated drug that targets and destroys the blood vessels that cause vision loss in wet AMD. In this treatment, Visudyne® is injected intravenously. When the drug reaches the eye, a low-intensity laser is directed to the region of blood vessel growth, activating the drug, which destroys the unhealthy vessels. PDT treatments are usually repeated, and may be performed in combination with other treatments such as Macugen® or Lucentis™.

3. Treatments Under Development

A Phase I human study of a gene therapy for the treatment of wet AMD has been completed. The treatment is an adenovirus-based delivery Pigment Epithelium Derived Factor (AdPEDF). The Ad-PEDF treatment involves the delivery of a gene that leads to the production of the protein PEDF, which helps keep photoreceptors healthy, thereby preserving vision. A Phase II study of AdPEDF is underway for treating patients with early to moderate wet AMD. FFB funded earlier, preclinical studies of PEDF.

Developed by Neurotech, ECT is a tiny capsule (6 millimeters) implanted into the eye. The capsule contains retinal cells that produce a vision-preserving protein called Ciliary Neurotrophic Factor (CNTF). The protein helps keep photoreceptors alive and healthy, thereby preserving vision. The ECT is currently in a Phase II human clinical trial for people with dry AMD. FFB funded earlier, preclinical studies of this therapy.

Developed by Genaera, EVIZON™ (squalamine lactate) is currently in a Phase III human study for the treatment of wet AMD. The drug works by blocking numerous factors that lead to the growth of unhealthy, vision-robbing blood vessels under the retina. Derived from the liver of the dogfish shark, EVIZON™ is a small molecule drug administered intravenously.

Othera's OT-551 is intended to supplement the eye's natural defense system against disease and injury. Permeating tissues at both the front of the eye (lens) and back of the eye (retina), researchers believe that OT-551 will provide antioxidant protection against both cataract and dry AMD. OT-551 is currently in a clinical study for the treatment of geographic atrophy (advanced dry AMD).

Developed by Alcon, Retaane® (anecortave acetate) is a modified steroid that has shown promise in reducing the risk of vision loss due to the growth of unhealthy blood vessels in wet AMD. Alcon is currently undertaking a clinical trial to determine if the drug prevents dry AMD from transforming into wet AMD.

Developed by Acuity Pharmaceuticals, siRNA (bevasiranib) is biological that silences the genes leading to the growth of unhealthy, vision-robbing blood vessels under the retina. The treatment has showed safety and efficacy in a Phase II study, and a Phase III clinical trial is planned.

Regeneron's wet AMD treatment, VEGF Trap, blocks the development of unhealthy blood vessels that lead to vision loss. A Phase I clinical trial has been successfully completed in patients with wet AMD utilizing systemic delivery, but eye injections are presently under development.

Other procedures undergoing under develop clinical trials include retinal transplantation, artificial retinas, retinal chips, and video relays using a camera and artificial chip, which in turn sends signals to the optic nerve. Humayan's device is modeled after the cochlear implant, which gives a rudimentary hearing capability to people who are deaf. Humayan and his colleagues are working on a new model of the device, which should improve the recipient's ability to perceive detail.

### VII. Examples

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Example 1

### Materials and Methods

For the logistic regression analyses, the inventors included age (in years), ever/never smoking (coded “1” for smokers, “0” for non-smokers), CFH Y402H (coded “1” for CC and CT genotypes, “0” for TT genotype), LOC387715 A69S (coded “1” for TT and GT genotypes, “0” for GG genotype), and the interaction between LOC387715 A69S and smoking (coded as the product of the genotype and smoking codes for each individual) in the model. Therefore, the logistic regression equation was:

g=β₀+β₁*Age+β₂*Y402H+β₃*A69S+β₄*Smoking+β₅*A69S-smoking interaction

and the probability of AMD for an individual was:

probability of AMD=e^{g}/(1+e^{g})

Once the probability of AMD was determined for each individual in the testing dataset, individuals with a probability greater than a particular threshold were classified as affected, and those below the threshold were classified as normal. These “model calls” can then be compared to the affection status assigned by a clinician and the sensitivity, specificity, and overall correct classification rate of the model can be determined. Changing the threshold for the probability of AMD will change the number of false positives and false negatives called by the model. Because there is no a priori reason to select a particular threshold value, the inventors chose to use 0.5 as a cut-off for their initial analyses.

For the if-then decision tree rules, the number of cases and controls with each particular susceptibility factor combination was calculated. If the ratio of cases to controls having this combination in the training dataset exceeded the total ratio of cases and controls, then individuals with the same combination in the testing dataset were called affected and vice versa. For example, there were 352 cases and 184 controls in the training dataset for a total ratio of 1.91. There were 21 cases and 4 controls that were non-smokers and had CC and GT genotypes at CFH Y402H and LOC387715 A69S respectively, for a ratio of 5.25. Therefore, all individuals with this combination in the testing dataset were classified as cases.

One advantage of the decision tree modeling compared to logistic regression is that there is no need to specify an arbitrary threshold value for classifying affection status. The major drawbacks with if-then decision tree rules are: 1) age cannot be included in the model without overly stratifying the datasets, and 2) large sample sizes are needed for each susceptibility combination to ensure stability of the model. The inventors have made progress in overcoming the age limitation by incorporating age into the decision tree model using codes of “1, 2, 3, or 4” based on the quantile that a given individual's age falls into.

Bayesian classification attempts to cluster similar individuals together based on their pattern of risk characteristics. The number of clusters may be preselected or not. To be useful in predicting AMD status, the clusters produced by Bayesian classification should correspond to case-control status. The inventors determined whether a particular cluster was associated with case or control status by determining the rules to maximize the total number of individuals correctly classified in the training dataset and then applying these rules to the testing dataset.

By combining approaches, the inventors can capitalize on the strengths of each method while minimizing errors.

The inventors evaluated the algorithm by calculating the sensitivity, specificity, overall correct classification rate, positive predictive value (PPV), and negative predictive value (NPV) according to the following equations:

Sensitivity=Probability(Affected_{model}|Affected_{reality})

Specificity=Probability(Normal_{model}|Normal_{reality})

Overall Classification Rate=(# Cases Correct+# Controls Correct)/(Total # of Individuals in the Testing Dataset)

Positive Preditive Value=(# Cases Correct)/(Total # Individuals Called a “Case” by the Model)

Negative Predictive Value=(# Controls Correct)/(Total # Individuals Called a “Control” by the Model)

### Example 2

### Results

The inventors began by estimating coefficients for each parameter in the model in a training dataset of 352 cases and 184 controls (Table 1), and then evaluating the success of the logistic model alone in the testing dataset of 89 cases and 48 controls (Table 2).

TABLE 1
Estimated Coefficients* for the Model Including
CFH Y402H, LOC387715 A69S, smoking, and LOC387715
A69S-smoking Interaction

| | Coeffi- | Standard | | | 95% Confidence | |
|---|---|---|---|---|---|---|
| | cient | Error | Z | p-value | Interval | |
| Age of Exam | 0.16 | 0.02 | 10.84 | <0.001 | 0.13 | 0.19 |
| Smoking | 0.15 | 0.34 | 0.45 | 0.650 | −0.51 | 0.81 |
| CFH Y402H | 0.90 | 0.26 | 3.48 | 0.001 | 0.39 | 1.40 |
| LOC387715 | | | | | | |
| A69S | 0.29 | 0.34 | 0.85 | 0.393 | −0.38 | 0.97 |
| A69S- | 0.60 | 0.46 | 1.32 | 0.186 | −0.29 | 1.50 |
| smoking | | | | | | |
| interaction | | | | | | |
| _constant | −12.08 | 1.13 | −10.66 | <0.001 | −14.30 | −9.86 |

*Note:
These coefficients will change depending on what specific factors are including in the model, and can easily be adapted as new susceptibility factors for AMD are discovered.

TABLE 2
Comparison of Logistic Regression to Actual AMD Status in the
Testing Dataset

Reality

| Model | A | N | |
|---|---|---|---|
| A | 76 | 23 | 99 |
| N | 13 | 25 | 38 |
| | 89 | 48 | 137 |

A = affected, N = normal
76/89 cases classified correctly = 85.4%
25/48 controls classified correctly = 52.1%
101/137 overall classified correctly = 73.7%
76/99 PPV = 76.8%
25/38 NPV = 65.8%

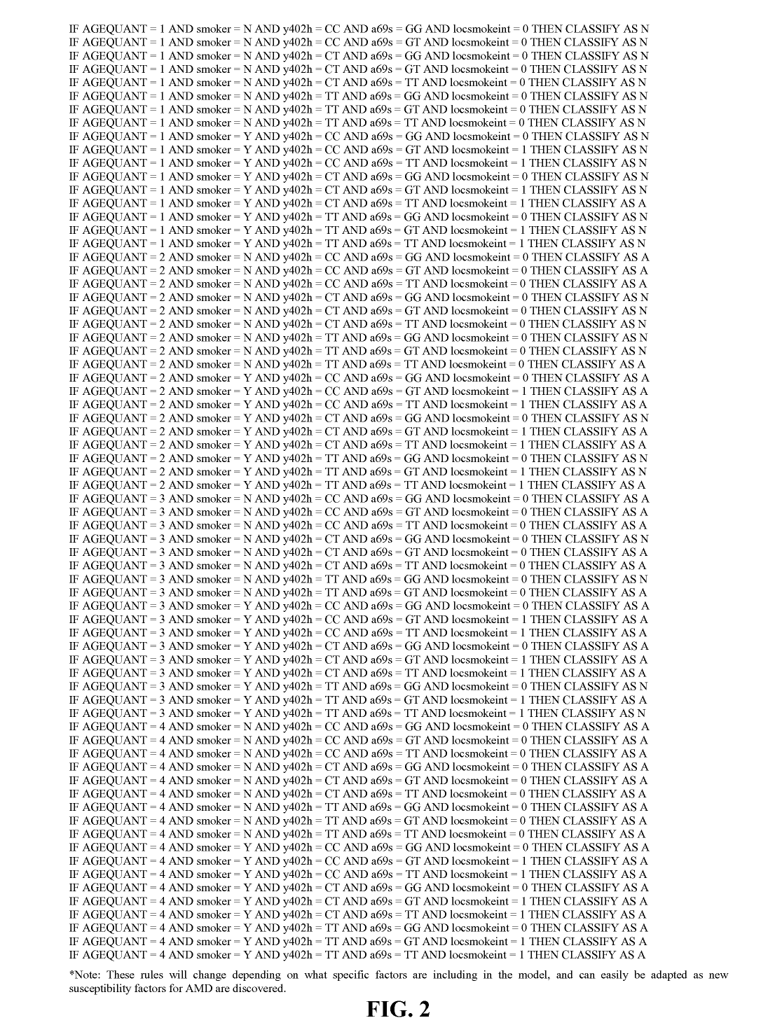

Next, the inventors used the same training dataset to build a set of decision tree rules (FIG. 2), and evaluated the sensitivity and specificity of this model (Table 3).

TABLE 3
Comparison of Decision Tree Rules to Actual AMD Status in the
Testing Dataset

Reality

| Model | A | N | |
|---|---|---|---|
| A | 69 | 12 | 81 |
| N | 19 | 36 | 55 |
| CNC* | 1 | 0 | 1 |
| | 89 | 48 | 137 |

*CNC = could not classify. This occurs when an individual in the testing dataset has a rare combination of risk factors that is not observed in the training dataset and cannot be classified. This happens very rarely.
69/89 cases classified correctly = 77.5%
36/48 controls classified correctly = 75.0%
105/137 overall classified correctly = 76.6%
69/81 PPV = 85.2%
36/55 NPV = 65.5%

Then, the inventors used Autoclass software to do a Bayesian classification analysis of these data, and evaluated this model (Table 4).

TABLE 4
Comparison of Bayesian Classification to Actual AMD Status in the
Testing Dataset

Reality

| Model | A | N | |
|---|---|---|---|
| A | 54 | 17 | 71 |
| N | 35 | 31 | 66 |
| | 89 | 48 | 137 |

54/89 cases classified correctly = 60.7%
31/48 controls classified correctly = 64.6%
85/137 overall classified correctly = 62.0%
54/71 PPV = 76.1%
31/66 NPV = 47.0%

Combining logistic regression, decision tree, and Bayesian classification approaches. By combining approaches, the inventors showed that they can maximize predictive accuracy (Table 5).

TABLE 5
Comparison of Consensus Predictions of the 3 Combined Methods to
Actual AMD Status in the Testing Dataset

| | % | % | | | % |
|---|---|---|---|---|---|
| | Cases | Controls | | | Overall |
| | Correct | Correct | PPV | NPV | Correct |
| Logistic | 85.4 | 52.1 | 76.8 | 65.8 | 73.7 |
| Decision Tree | 77.5 | 75.0 | 85.2 | 65.5 | 76.6 |
| Bayesian Classification | 60.7 | 64.6 | 76.1 | 47.0 | 62.0 |
| Consensus 2/3 Methods | 82.0 | 66.7 | 82.0 | 66.7 | 76.6 |
| Consensus 3/3 Methods - | 48.3 | 35.4 | NR* | NR* | 43.8 |
| all | | | | | |
| Consensus 3/3 Methods - | 87.8 | 77.3 | 89.6 | 73.9 | 84.5 |
| only inds with perfect | | | | | |
| agreement | | | | | |

*NR = not reported.
PPV and NPV are not reported for the consensus 3/3 methods - all because they cannot be meaningfully interpreted for this scenario.

When requiring that all 3 methods agree on the affection status prediction, overall only 43.8% of individuals in the testing dataset are classified correctly. However, this is due to the large proportion of individuals for which not all 3 methods agreed (48.2%). Excluding those individuals, 84.5% of individuals could be correctly classified (87.8 of cases and 77.3% of controls), a huge gain compared to the best single method (decision trees 76.6%). The gains in PPV and NPV are also substantial. However, these gains come at the expense of not reporting a consensus prediction for 48.2% of individuals. Using a consensus prediction when any 2 of the 3 methods agree does not result in any gain over using the single best method alone (76.6% overall correct for both decision trees and consensus 2/3 methods). The overall classification rate, sensitivity, and specificity for this algorithm were very high considering the complexity of the AMD phenotype. The inventors are currently working to increase the range of individuals that the algorithm can classify and determining the optimal way to combine the 3 approaches, for example, by weighting the result of one of the three methods depending on the weights of the specific variables. The value of this algorithm is its flexibility in using multiple different variables in different arrangements to maximize predictive ability. The accuracy, sensitivity, and specificity of this algorithm can certainly be at least marginally increased by including other known AMD susceptibility factors in the future (e.g., C3 R102G, mito4917, VEGF variants). The high accuracy in a subset of potential AMD sufferers and the flexibility to easily incorporate new susceptibility factors as knowledge of the AMD phenotype grows make this algorithm a valuable tool.

All of the compositions and/or methods disclosed and claimed herein can be made and executed without undue experimentation in light of the present disclosure.

While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. References

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,582,788
U.S. Pat. No. 4,656,127
U.S. Pat. No. 4,659,774
U.S. Pat. No. 4,682,195
U.S. Pat. No. 4,683,194
U.S. Pat. No. 4,683,195
U.S. Pat. No. 4,683,202
U.S. Pat. No. 4,800,159
U.S. Pat. No. 4,816,571
U.S. Pat. No. 4,883,750
U.S. Pat. No. 4,938,948
U.S. Pat. No. 4,946,773
U.S. Pat. No. 4,959,463
U.S. Pat. No. 5,021,236
U.S. Pat. No. 5,141,813
U.S. Pat. No. 5,196,066
U.S. Pat. No. 5,264,566
U.S. Pat. No. 5,279,721
U.S. Pat. No. 5,428,148
U.S. Pat. No. 5,554,744
U.S. Pat. No. 5,574,146
U.S. Pat. No. 5,602,244
U.S. Pat. No. 5,645,897
U.S. Pat. No. 5,705,629
U.S. Pat. No. 5,840,873
U.S. Pat. No. 5,843,640
U.S. Pat. No. 5,843,651
U.S. Pat. No. 5,843,663
U.S. Pat. No. 5,846,708
U.S. Pat. No. 5,846,717
U.S. Pat. No. 5,846,726
U.S. Pat. No. 5,846,729
U.S. Pat. No. 5,849,481
U.S. Pat. No. 5,849,483
U.S. Pat. No. 5,849,486
U.S. Pat. No. 5,849,487
U.S. Pat. No. 5,851,770
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,861,244
U.S. Pat. No. 5,863,732
U.S. Pat. No. 5,863,753
U.S. Pat. No. 5,866,331
U.S. Pat. No. 5,866,337
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,779
U.S. Pat. No. 5,919,626
U.S. Pat. No. 5,919,630
U.S. Pat. No. 5,925,517
U.S. Pat. No. 5,925,525
U.S. Pat. No. 5,928,862
U.S. Pat. No. 5,928,869
U.S. Pat. No. 5,928,870
U.S. Pat. No. 5,929,227
U.S. Pat. No. 5,932,413
U.S. Pat. No. 5,935,791
U.S. Pat. No. 5,952,174
Abbondanzo et al., Breast Cancer Res. Treat., 16:182(151), 1990.
Alge et al., Invest. Ophthalmol. Vis. Sci., 43(11):3575-3582, 2002.
Allikmets et al., Nat. Genet., 15:236-246, 1997.
Allred et al., Arch. Surg., 125(1):107-113, 1990.
Anderson et al., Nature, 290(5806):457-465, 1981.
Atherton et al., Biol. Reprod., 32(1):155-171, 1985.
Attebo et al., Ophthalmol., 103:357-364, 1996.
Ausubel et al., In: Current Protocols in Molecular Biology, John, Wiley & Sons, Inc, NY, 1989.
Ballinger et al., Exp. Eye Res., 68(6):765-772, 1999.
Beatty et al., Surv. Ophthalmol., 45(2):115-134, 2000.
Bellus, J., Macromol. Sci. Pure Appl. Chem., A31(1): 1355-1376, 1994.
Berberian et al., Science, 261:1588-1591, 1993.
Bird et al., Surv. Ophthalmol., 39:367-374, 1995.
Brown et al. Immunol. Ser., 53:69-82, 1990.
Brown et al., Am. J. Hum. Genet., 60: 381-387, 1997.
Browne et al., J. Infect. Dis., 167(1):21-29, 1993.
Campbell, In: Monoclonal Antibody Technology, Laboratory Techniques in Biochemistry and Molecular Biology, Burden and Von Knippenberg (Eds.), Elsevier, Amsterdam, 13:71-74/75-83, 1984.
Cann et al., Genetics, 106(3):479-499, 1984.
Centers for Disease Control and Prevention (CDC), 2002
Cleary et al., J. Biol. Chem., 269(29):18747-9, 1994.
Conley et al., Hum. Mol. Genet., 14(14):1991-2002, 2005.
Cui et al., J. Pharmacol. Exp. Ther., 280(3):1228-1234, 1997.
Dalakas et al., Lab. Invest., 81(11):1537-1544, 2001.
de Arruda et al., Expert. Rev. Mol. Diagn., 2:487-496, 2002.
De Benedictis et al., FASEB J., 13(12):1532, 1999.
De Jager et al., Semin. Nucl. Med., 23(2):165-179, 1993.
Dholakia et al., J. Biol. Chem., 264(34):20638-20642, 1989.
DiMauro and Schon, N. Engl. J. Med., 348:2656-2668, 2003.
Doolittle and Ben-Zeev, Methods Mol. Biol., 109:215-237, 1999.
Dunaief et al., Arch. Ophthalmol., 120:1435-1442, 2002.
Edwards et al., Science, 308(5720):421-424, 2005.
European Patent 201,184
European Patent 237,362
European Patent 258,017
European Patent 266,032
European Patent 320 308
European Patent 329 822
European Patent Appln. 50,424
European Patent Appln. 84,796
Fauser et al., Biochem. Biophys. Res. Commun., 295(2):342-7, 2002.
Ferris et al., Arch. Ophthalmol., 102(11):1640-1642, 1984.
Fisher et al., Hum. Mol. Genet., 14(15):2257-2264, 2005.
French Patent 2,650,840
Froehler et al., Nucleic Acids Res., 14(13):5399-5407, 1986.
Frohman, In: PCR Protocols: A Guide To Methods And Applications, Academic Press, N.Y., 1990.
Garcia-Ruiz et al., J. Biol. Chem., 272:11369-11377, 1997.
Gefter, et al., Somatic Cell Genet., 3:231-236, 1977.
Giacchetti et al., J. Med. Gen., 41:293-295, 2004.
Goding, In: Monoclonal Antibodies: Principles and Practice, 2d ed., Academic Press, FL., 60-66, and 71-74, 1986.
Great Britain Appln. 2 202 328
Gulbis and Galand, Hum. Pathol., 24(12):1271-1285, 1993.
Hageman et al., Proc. Natl. Acad. Sci. USA, 102(20):7227-7232, 2005.
Haines, Science, 308(5720):419-421, 2005.
Halushka et al., Nat. Genet., 22(3):239-247, 1999.
Hammond et al., Ophthalmology, 109(4):730-736, 2002.
Heiba et al., Genet. Epidemiol., 11(1):51-67, 1994.
Herrnstadt and Howell, Mitochondrion., 4:791-798, 2004.
Hoffman et al., Am. J. Hum. Genet., 60:1539-1542, 1997.
Hofmann et al., Genomics, 39(1):8-18, 1997.
Howell et al., Genetics, 140(1):285-302, 1995.
Hughes et al., PLoS. Med. 4, e355, 2007.
Hulgan et al., AIDS, 19(13):1341-1349, 2005.
Inazuka et al., Genome Res, 7(11):1094-1103, 1997.
Innis et al., Proc. Natl. Acad. Sci. USA, 85(24):9436-9440, 1988.
Jakobsdottir et al., Am. J. Hum. Genet., 77(3):389-407, 2005.
Jin et al., Curr. Eye Res., 23(1):11-9, 2001.
Johnson et al., Nat. Genet., 29(2):233-237, 2001.
Kang et al., Science, 240:1034-1036, 1988.
Ke and Cardon, Bioinformatics, 19(2):287-288, 2003.
Kelleher et al., Clin. Ther., 21(7):1182-1192, 1999.
Khatoon et al., Ann. of Neurology, 26, 210-219, 1989.
King et al., J. Biol. Chem., 269, 10210-10218, 1989.
Kirchner et al., Neurotoxicology, 21(4):441-445, 2000.
Klayer et al., Arch. Ophthalmol., 116(12):1646-1651, 1998.
Klein and Francis, Ophthalmol. Clin. N. Am., 16:567-574, 2003.
Klein et al., Science, 308(5720):385-389, 2005.
Kohler and Milstein, Eur. J. Immunol., 6:511-519, 1976.
Kohler and Milstein, Nature, 256:495-497, 1975.
Kohler et al., Methods Enzymol., 178:3, 1989.
Komher et al., Nucl. Acids. Res. 17:7779-7784, 1989.
Kreier et al., In: Infection, Resistance and Immunity, Harper & Row, New York, 1991.
Kuppuswamy et al., Proc. Natl. Acad. Sci. USA, 88:1143-1147, 1991.
Kwoh et al., Proc. Natl. Acad. Sci. USA, 86:1173, 1989.
Kwok and Chen, Curr Issues Mol. Biol., April; 5(2):43-60, 2003.
Kwok et al., Methods Mol. Biol., 212:71-84, 2003.
Kwok et al., Genomics, 23(1):138-144, 1994.
Kwok, Annu. Rev. Genomics Hum. Genet., 2:235-258, 2001.
Landegren, et al., Science 241:1077-1080, 1988.
Lenert et al., Science, 248(4963):1639-1643, 1990.
Liang et al., Exp. Eye Res., 78(6):1069-1067, 2004.
Lotery et al., Invest. Ophthalmol. Vis. Sci., 41:1291-1296, 2000.
Lu et al., Biopolymers, 73:606-613, 2004.
Mailer et al., Nat. Genet., 38, 1055-1059, 2006.
Man et al., J. Med. Genet., 39:162-169, 2002.
Mancuso et al., Neurosci. Lett., 371:158-162, 2004.
Maxam et al., Proc. Natl. Acad. Sci. USA, 74:560, 1977.
Mishmar et al., Proc. Natl. Acad. Sci. USA, 100:171-176, 2003.
Morb. Mortal Wkly. Rep. (MMWR), 53:1069-1071, 2004.
Moriarty-Craige et al., Am. J. Ophthalmol., 140(6):1020-1026, 2005.
Mullis et al., Cold Spring Harbor Symp. Quant. Biol. 51:263-273, 1986.
Nakamura et al., In: Handbook of Experimental Immunology (4^{th} Ed.), Weir et al., (eds). 1:27, Blackwell Scientific Publ., Oxford, 1987.
Nichols et al., Nat. Genet., 3:202-207, 1993.
Nickerson et al., Proc. Natl. Acad. Sci. USA, 87:8923-8927, 1990.
Nyren et al., Anal. Biochem. 208:171-175, 1993.
Ohara et al., Proc. Natl. Acad. Sci. USA, 86:5673-5677, 1989.
Ohia et al., Mutat. Res., 579(1-2):22-36, 2005.
Orth and Schapira, Neurochem. Int., 40:533-541, 2002.
O'Shannessy et al., J. Immun. Meth., 99, 153-161, 1987.
Owens & Haley, J. Biol. Chem., 259:14843-14848, 1987.
PCT Appln. PCT/US87/00880
PCT Appln. PCT/US89/01025
PCT Appln. WO 88/10315
PCT Appln. WO 89/06700
PCT Appln. WO 91/02087
PCT Appln. WO 92/15712
Penta et al., Mutat. Res., 488:119-133, 2001.
Petrukhin et al., Nat. Genet., 19:241-247, 1998.
Postel et al., Am. J. Ophthalmol., 139(5):820-825, 2005.
Potter & Haley, Meth. in Enzymol., 91, 613-633, 1983.
Prezant et al., Hum. Mutat., 1:159-164, 1992.
Rivera et al., Hum. Mol. Genet., 14(21):3227-3236, 2005.
Robinson, Biochim. Biophys. Acta., 1364:271-286, 1998.
Ross et al., Exp. Gerontol., 36:1161-1178, 2001.
Ross et al., Exp. Gerontol., 38(4):397-405, 2003.
Ruiz-Pesini et al., Am. J. Hum. Genet., 67:682-696, 2000.
Sambrook and Russell, Molecular Cloning: A Laboratory Manual 3^{rd} Ed., Cold Spring Harbor Laboratory Press, 2001.
Sanger et al., J. Molec. Biol., 94:441, 1975.
Sasso et al., J. Immunol., 142:2778-2783, 1989.
Schmidt et al., Am J Hum Genet. 78:852-864, 2006.
Schmidt et al., Mol. Vis., 6:287-293, 2000.
Seddon et al., Am. J. Ophthalmol., 123:199-206, 1997.
Seddon et al., Jama, 272:1413-1420, 1994.
Shoffner et al., Genomics, 17:171-184, 1993.
Shorki et al., J. Immunol., 146:936-940, 1991.
Silvermann et al., J. Clin. Invest., 96:417-426, 1995.
Smeitink and van den Heuvel, Am. J. Hum. Genet., 4:1505-1510, 1999.
Smith et al., Ophthalmol., 108:697-704, 2001.
Sokolov, Nucl. Acids Res. 18:3671, 1990.
Sommer et al., N. Engl. J. Med., 325:1412-1417, 1991.
Spraul et al., Invest. Ophthalmol. Vis. Sci., 37(13):2724-2735, 1996.
Stevens et al., Biotechniques, 34:198-203, 2003.
Stone et al., Nat. Genet., 20:328-329, 1998.
Stone et al., Nat. Genet., 22:199-202, 1999.
Syvanen et al., Genomics 8:684-692, 1990.
Taillon-Miller et al., Genome Res, 8(7):748-754, 1998.
The Eye Diseases Prevalence Research Group. Prevalence of Age-Related macular Degeneration in the United States. Arch. Ophthalmol., 122:564-572, 2004.
Tielsch et al., N. Engl. J. Med., 332:1205-1209, 1995.
Torroni and Wallace, J. Bioenerg. Biomembr., 26:261-271, 1994.
Torroni et al., Am. J. Hum. Genet., 55:760-776, 1994.
Torroni et al., Am. J. Hum. Genet., 60(5):1107-1121, 1997.
Torroni et al., Am. J. Hum. Genet., 60:1107-1121, 1997.
Torroni et al., Genetics, 144(4):1835, 1996.
Torroni et al., Genetics, 144:1835-1850, 1996.
Ugozzoll et al., GATA, 9:107-112, 1992.
van der Walt et al., Am. J. Hum. Genet., 72:804-811, 2003.
van der Walt et al., Neurosci. Lett., 365:28-32, 2004.
Walker et al., Proc. Natl. Acad. Sci. USA, 89:392-396 1992.
Wallace et al., Gene, 238:211-230, 1999.
Wallace, Proc. Natl. Acad. Sci. USA, 91(19):8739-8746, 1994.
Wallace, Science, 283:1482-1488, 1999.
Weber et al., Nat. Genet., 8(4):352-356, 1994.
Weeks et al., Am. J. Hum. Genet., 75(2):174-189, 2004.
Zareparsi et al., Am. J. Hum. Genet., 77(1):149-153, 2005.
Zhang et al., Nat. Genet., 27:89-93, 2001.

1. A method of screening an individual for susceptibility to age-related macular degeneration (AMD) comprising assessing (a) the individual's structure and/or function of a Complement factor H(CHF) protein; (b) the individual's structure at LOC387715/ARMS2; and (c) the interaction between the individual's structure at LOC387715/ARMS2 and the individual's smoking history.

2. The method of claim 1, wherein (a) comprises assessing the structure of a CFH-encoding nucleic acid from said individual.

3. The method of claim 2, wherein assessing comprises determining the CFH-encoding nucleic acid sequence for all or a portion of a CFH-encoding RNA.

4. The method of claim 3, wherein assessing comprises determining the portion of the CFH-encoding nucleic acid sequence corresponding to position Y402.

5. The method of claim 4, further comprising amplifying all or part of said individual's CFH-encoding nucleic acid.

6. The method of claim 5, wherein amplifying comprises polymerase chain reaction (PCR).

7. The method of claim 3, wherein determining the sequence CFH-encoding nucleic acid sequence comprises determining the sequence of a nucleic acid that is in linkage disequilibrium with position Y402.

8. The method of any of claim 7, wherein determining the sequence comprises differential hybridization.

9. The method of claim 1, wherein (a) comprises assessing the function of a CFH protein from said individual.

10. The method of claim 1, wherein (b) comprises assessing the presence or absence of a T allele at rs10490924.

11. The method of claim 10, wherein assessing comprises sequencing of rs10490924.

12. The method of claim 11, further comprising amplifying all or part of said individual's nucleic acid at rs10490924.

13. The method of claim 12, wherein amplifying comprises polymerase chain reaction (PCR).

14. The method of claim 10, wherein assessing comprises determining the sequence of a nucleic acid that is in linkage disequilibrium with rs10490924.

15. The method of any of claim 10, wherein assessing comprises differential hybridization.

16. The method of claim 1, wherein (c) comprises a product of the codes for individual susceptibility factors.

17. The method of claim 1, further comprising assessing genetic variation in said individual's mtDNA.

18. The method of claim 17, wherein assessing comprises determining the mtDNA sequence at positions corresponding to the MTND1*LHON4216C and/or MTND2*LHON4917G alleles.

19. The method of claim 18, wherein determining the sequence of said individual's mtDNA comprises sequencing of all or a portion of a corresponding RNA.

20. The method of claim 19, further comprising amplifying all or part of said individual's mtDNA.

21. The method of claim 20, wherein amplifying all or part of said individual's mtDNA comprises polymerase chain reaction (PCR).

22. The method of claim 18, wherein determining the sequence of said individual's mtDNA at positions corresponding to the MTND1*LHON4216C and/or MTND2*LHON4917G alleles by determining the sequence of a mitochondrial RNA that comprises a polymorphism that is in linkage disequilibrium with the MTND1*LHON4216C and/or MTND2*LHON4917G alleles.

23. The method of claim 22, further comprising amplifying all or part of said individual's mtDNA.

24. The method of claim 23, wherein amplifying all or part of said individual's mtDNA comprises polymerase chain reaction (PCR).

25. The method of claim 18, wherein assessing the presence or absence of the MTND1*LHON4216C and/or MTND2*LHON4917G alleles comprises assessing the presence or absence of the MTND1*LHON4216C and/or MTND2*LHON4917G alleles in said individual's maternal blood relative's mtDNA.

26. The method of claim 18, wherein assessing the presence or absence of the MTND1*LHON4216C and/or MTND2*LHON4917G alleles comprises differential hybridization.

27. The method of claim 1, further comprising assessing genetic variation in said individual's C2/CFB gene cluster.

28. The method of claim 1, further comprising assessing genetic variation in said individual's C3 gene locus.

29. The method of claim 1, further comprising assessing genetic variation in said individual's VEGF gene locus.

30. The method of claim 1, wherein said AMD is wet AMD.

31. The method of claim 1, wherein said AMD is dry AMD.

32. A method for determining the need for prophylactic treatment for age-related macular degeneration (AMD) comprising assessing (a) the individual's structure and/or function of a Complement factor H(CHF) protein; (b) the individual's structure at LOC387715/ARMS2; and (c) the interaction between the individual's structure at LOC387715/ARMS2 and the individual's smoking history.

33. The method of claim 32, further comprising assessing genetic variation in said individual's mtDNA.

34. The method of claim 32, further comprising assessing genetic variation in said individual's C2/CFB gene cluster, genetic variation in said individual's C3 gene locus, and/or genetic variation in said individual's VEGF gene locus.

35. A kit comprising, in a suitable container means, at least one nucleic acid for determining:
(i) the presence or absence of one or more of (a) a T allele at rs10490924 or (b) a mutation in the Complement H gene; and
(ii) the structure at LOC387715/ARMS2.

36. The kit of claim 35, further comprising (iii) a nucleic acid for determining the presence or absence of the MTND1*LHON4216C allele and/or at least one nucleic acid for determining the presence or absence of the MTND2*LHON4917G allele; (iv) a nucleic acid for determining a sequence with a C3 gene locus; (v) a nucleic acid for determining a sequence with a C2/CFB gene cluster; and/or (vi) a nucleic acid for determining a sequence within an VEGF gene locus.
